(19) United States
(12) Patent Application Publication
(10) Pub. No.: US 2026/0232550 A1
(43) Pub. Date: Aug. 13, 2026

(54) TRANSPARENT ALCOHOL-FREE BASE FOR FRAGRANCE COMPOSITIONS

(71) Applicant: INTERNATIONAL FLAVORS & FRAGRANCES INC., NEW YORK, NY (US)

(72) Inventors: ANDREA NOEL KROENIG, Hoboken, NJ (US); Stephanie WOIMANT, Conflans Sainte Honorine (FR); Estelle LOING, Brooklyn, NY (US); Hawa CONTEH, New York, NY (US)

(21) Appl. No.: 19/155,322

(22) PCT Filed: Feb. 12, 2024
(86) PCT No.: PCT/US2024/015365

§ 371 Date: Aug. 11, 2025

Related U.S. Application Data
(60) Provisional application No. 63/485,285, filed on Feb. 16, 2023.

(51) Int. Cl. A61K 8/34; A61K 8/36; A61K 8/49; A61Q 13/00

(52) U.S. Cl. CPC A61K 8/345; A61K 8/34; A61K 8/361; A61K 8/4973; A61Q 13/00; A61K 2800/30

(57) ABSTRACT

A transparent alcohol-free base for fragrances is provided. A fragrance composition and consumer product including the transparent alcohol-free base are also provided.

### BACKGROUND

In the perfume industry there has long been a demand for aqueous, ethanol-free perfume oil compositions. Ethanol-containing solutions of perfume oils are certainly widely used, but such compositions have various drawbacks, which are sometimes unacceptable to the consumer. Objections to the use of ethanol in perfume oil compositions are based for example on a risk of skin irritations attributable to ethanol, religious reasons, or the carcinogenic potential of ethanol, as well as legal regulations on avoidance of volatile organic substances (VOCs). This contrasts with the acceptance of aqueous formulations, which are nonflammable and are generally regarded as harmless by consumers. Accordingly, in the past the perfume industry has already attempted to develop suitable, ethanol-free perfume oil compositions. However, so far it has not been possible to provide ethanol-free perfume oil compositions that have been accepted (without qualification) by the consumer.

Ethanol-free perfume oil compositions known in the prior art as a rule contain-along with perfume oil(s), water and optionally preservative(s) and one or a plurality of surfactants, which are used for solubilizing the perfume oil or oils. Surfactants can, however, cause skin irritations and as a rule result in the perfume composition feeling sticky, in the opinion of consumers.

US 2003/0186836 A1 describes alcohol-free, aqueous perfume oil compositions, which contain isoprene glycol (3-methyl-1,3-butanediol) as solvent (instead of ethanol).

WO 2005/123028 A1 describes an ethanol-free, perfumed, aqueous microemulsion, which comprises a vicinal diol as solvent. According to WO 2005/123028 A1, surfactants are only contained optionally in the microemulsion.

U.S. Pat. No. 8,343,521 B2 discloses a ethanol-free microemulsion composition composed of a fragrance material, wherein the fragrance material is present in an amount greater than 5% and less than 50% by weight; a solvent, wherein said solvent is 1,2-hexanediol or 1,2-heptanediol; and an aqueous medium, wherein the composition does not contain surfactants.

U.S. Pat. No. 8,513,180 B2 describes an ethanol-free perfume oil microemulsion composed of water, at least one vicinal diol, and one or two solvents for reducing stickiness, wherein the solvents include isobutyric acid-1-hydroxy-2,2,4-trimethyl-3-pentyl ester or isobutyric acid-3-hydroxy-2,2,4-trimethyl-1-pentyl ester.

There is still interest in providing ethanol-free fragrance compositions, which are transparent and can be used in varied applications in the perfume industry. The present invention addresses this need in the art.

### SUMMARY OF THE INVENTION

This invention is a transparent alcohol-free base composed of (i) at least one solubilizer, said solubilizer being selected from the group of polysorbate 20, sodium olivate, Glycereth Cocoate and polyethylene glycol (16) macadamia glycerides; (ii) at least two emollients, said emollients comprising isopentyldiol and 3-methoxy-3-methyl-1-butanol; and (iii) at least two solvents, said solvents comprising water and pentylene glycol.

The invention is also a fragrance composition composed the transparent alcohol-free base and at least one fragrance ingredient. In some aspects, the fragrance composition is composed of (i) 3 to 30 wt. %, such as between 3 to 25 wt. %, between 5 to 30 wt. %, or between 5 to 25 wt. % 5 to 20 wt. % of at least one fragrance ingredient; (ii) 5 to 30 wt. % at least one solubilizer, said solubilizer being selected from the group of polysorbate 20, sodium olivate, Glycereth Cocoate and polyethylene glycol (16) macadamia glycerides; (iii) 3 to 23 wt. % at least two emollients, said emollients comprising isopentyldiol and 3-methoxy-3-methyl-1-butanol; and (iv) 40 to 95 wt. % at least two solvents, said solvents comprising water and pentylene glycol. In certain aspects, the fragrance composition is composed of (i) 3 to 30 wt. %, such as between 3 to 25 wt. %, between 5 to 30 wt. %, or between 5 to 25 wt. % or between 5 to 20 wt. % of at least one fragrance ingredient; (ii) 5 to 30 wt. % polysorbate 20, sodium olivate, Glycereth Cocoate or polyethylene glycol (16) macadamia glycerides; (iii) 2 to 15 wt. % isopentyldiol; (iv) 1 to 8 wt. % 3-methoxy-3-methyl-1-butanol; (v) 15 to 35 wt. % pentylene glycol; and (vi) 25 to 60 wt. % water. A method for preparing the fragrance composition is also provided.

The invention further provides a consumer product including the fragrance composition, wherein said consumer product is a fine fragrance, solid perfume, eau de parfum, parfum, cologne, alcohol-free perfume, perfume powder, perfume wax, perfume emulsion, powder foundation, or eau de toilette product, deodorant, air freshener, room spray or laundry spray.

### DETAILED DESCRIPTION OF THE INVENTION

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, use of “a” or “an” are used to describe elements and components described herein. This is done merely for convenience and to give a general sense of the scope of the invention. This description should be read to include one or at least one and the singular also includes the plural unless it is obvious that it is meant otherwise.

Unless otherwise defined, all technical and scientific terms used herein have the same meaning as commonly understood by one of ordinary skill in the art to which this invention belongs. In case of conflict, the present specification, including definitions, will control. Although methods and materials similar or equivalent to those described herein can be used in the practice or testing of embodiments of the present invention, suitable methods and materials are described below. In addition, the materials, methods, and examples are illustrative only and not intended to be limiting.

The present disclosure illustratively described herein suitably may be practiced in the absence of any element or elements, limitation or limitations that are not specifically disclosed herein. While compositions and methods are described herein in terms of “comprising” various components or steps, the compositions and methods also can “consist essentially of” or “consist of” the various components or steps, unless stated otherwise.

A new combination of cosmetic-approved ingredients has been identified, which provides a non-tacky, quick-drying, alcohol-free fragrance base with no residual film. The fragrance base is transparent allowing for the design of various fragrance compositions using non-restrictive fragrance ingredient guidelines. Advantageously, the fragrance base can be dosed with between 3 to 30 wt. %, such as between 3 to 25 wt. %, between 5 to 30 wt. %, or between 5 to 25 wt. % or between 5 wt. % and 20 wt. % fragrance ingredients and is particularly useful in the preparation of a sprayable alcohol-free compositions such as Eau De Toilette.

More specifically, this invention is a transparent alcohol-free base and use of the same for preparing fragrance compositions, wherein the base is composed of:
(i) at least one solubilizer, said solubilizer being selected from the group of polysorbate 20, sodium olivate, Glycereth Cocoate and polyethylene glycol (16) macadamia glycerides;
(ii) at least two emollients, said emollients comprising isopentyldiol and 3-methoxy-3-methyl-1-butanol; and
(iii) at least two solvents, said solvents comprising water and pentylene glycol.

The base of this invention is transparent in that transmits light without appreciable scattering so that objects lying beyond are seen clearly. Alternatively stated, the fragrance base of the invention is clear and therefore is not cloudy or opaque. Likewise, a fragrance composition including the fragrance base is transparent.

As indicated, the base is “alcohol-free” and, in the context of the present invention, preferably includes substantially no alcohol, in particular no ethanol, or at least less than 0.01 wt. % alcohol, preferably less than 0.001 wt. % alcohol, relative to the total weight of the base or fragrance composition of this invention.

In certain aspects, the base is formulated as a microemulsion. The term “microemulsion” denotes, in the context of the present invention, a macroscopically homogeneous, optically transparent and thermodynamically stable mixture.

The base of this invention includes at least one solubilizer. A “solubilizer” refers to an agent that increases the solubility of another substance. In some aspects, the solubilizer is polysorbate 20 (CAS No. 9005-64-5), sodium olivate (CAS No. 61789-88-6), Glycereth Cocoate (CAS No. 68201-46-7), polyethylene glycol (16) macadamia glycerides (CAS No. 220459-99-4), or a combination thereof. In some aspects, the solubilizer is polysorbate 20.

The base of this invention further includes at least two emollients. An “emollient” refers to a substance that helps soothe, soften, and increase moisture levels, especially in the skin. In certain aspects the at least two emollients of the present fragrance base are isopentyldiol (a.k.a., isoprene glycol; CAS No. 2568-33-4) and 3-methoxy-3-methyl-1-butanol (CAS No. 56539-66-3).

The base of this invention also includes at least two solvents. A “solvent” refers to a substance that dissolves a solute, resulting in a solution. In certain aspects the solvents of the present fragrance base are water, butylene glycol (CAS No. 6290-03-5) and pentylene glycol (1,2-pentanediol; CAS No. 5343-92-0).

For the purposes of the present invention the fragrance base does not include one or more of glycerol, hexylene glycol, isobutyric acid-1-hydroxy-2,2,4-trimethyl-3-pentyl ester, or isobutyric acid-3-hydroxy-2,2,4-trimethyl-1-pentyl ester).

In some aspects, the transparent alcohol-free base comprises, consists of or consists essentially of between about 15 wt. % and 25 wt. % solubilizer component relative to the total weight of the base; between 5 wt. % and 10 wt. % isopentyldiol relative to the total weight of the base; between 3 wt. % and 8 wt. % 3-methoxy-3-methyl-1-butanol relative to the total weight of the base; between 35 wt. % and 45 wt. % water relative to the total weight of the base; and between 20 wt. % and 30 wt. % pentylene glycol relative to the total weight of the base. In some aspects, the base further includes between about 1 wt % and 10 wt % dipropylene glycol (DPG).

In other aspects, the transparent alcohol-free base is composed of between about 15 wt. % and 25 wt. % of either polysorbate 20, sodium olivate, Glycereth Cocoate, polyethylene glycol (16) macadamia glycerides, or a combination thereof, relative to the total weight of the base; between 5 wt. % and 10 wt. % isopentyldiol relative to the total weight of the base; between 3 wt. % and 8 wt. % 3-methoxy-3-methyl-1-butanol relative to the total weight of the base; between 35 wt. % and 45 wt. % water relative to the total weight of the base; and between 20 wt. % and 30 wt. % pentylene glycol relative to the total weight of the base. In some aspects, the base further includes between about 1 wt % and 10 wt % dipropylene glycol (DPG).

In further aspects, the present invention provides a fragrance composition composed of the fragrance base in combination with one or more fragrance ingredients. As used herein, the term “fragrance composition,” “fragrance formulation,” “perfume composition,” “fragrance accord,” or “perfume accord” is a composition composed of one or more fragrances ingredients to impart a pleasant odor or cover a malodor.

In some aspects, the invention provides a fragrance composition composed of at least one fragrance, at least one solubilizer, at least two emollients, and at least two solvents. In certain aspects, the invention provides a fragrance composition comprising, consisting essentially of or consisting of at least one fragrance; at least one solubilizer, said solubilizer being selected from the group of polysorbate 20, sodium olivate, Glycereth Cocoate and polyethylene glycol (16) macadamia glycerides; at least two emollients, said emollients being selected from the group of isopentyldiol and 3-methoxy-3-methyl-1-butanol; and at least two solvents, said solvents being selected from the group of water and pentylene glycol.

As used herein, the terms “perfume,” “fragrance” “fragrance material” and “fragrance ingredient” are used interchangeably to designate the component in the fragrance composition that is capable of imparting or modifying the odor of a substrate, e.g., skin. A “fragrance ingredient” can encompass any suitable perfume raw material for fragrance uses, including materials such as, for example, alcohols, aldehydes, ketones, esters, ethers, acetates, nitriles, terpene hydrocarbons, nitrogenous or sulfurous heterocyclic compounds and essential oils. Non-limiting examples of the fragrance materials can also include pro-fragrances such as acetal pro-fragrances, ketal pro-fragrances, ester pro-fragrances, hydrolyzable inorganic-organic pro-fragrances, and mixtures thereof. The fragrance materials may be released from the pro-fragrances in a number of ways. For example, the fragrance may be released as a result of simple hydrolysis, or by a shift in an equilibrium reaction, or by a pH change, or by enzymatic release. Log P and water solubility determine how stable full fragrances are in the base. High dosages of ingredients with lower log P values and high-water solubility yield the best results in the bases described herein. Individual fragrance ingredients are readily available from commercial sources such as International Flavor & Fragrances (New York, NY), Givaudan Fragrances Corp. (East Hanover, NJ), Symrise (Branchburg, NJ), and Firmenich (Princeton, NJ).

The fragrance composition may include at least 1, 2, 3, 4, 5, 6, 7, 8, 9, 10 or more fragrance ingredients. In some aspects, the fragrance composition includes at least 10, at least 15, at least 20, at least 25, at least 30, at least 35, at least 40, at least 45, and/or at least 50 additional fragrance ingredients. The more fragrance raw materials that make up a fragrance composition, the more complex the fragrance composition is considered to be.

Fragrance ingredients that can be formulated with the base of this invention may include:
(i) essential oils, concretes, absolutes, resins, resinoids, balsams, tinctures such as for example ambergris tincture, amyris oil, angelica seed oil, angelica root oil, aniseed oil, valerian oil, basil oil, tree moss absolute, bay oil, armoise oil, benzoe resinoid, bergamot oil, beeswax absolute, birch tar oil, bitter almond oil, savory oil, buchu leaf oil, cabreuva oil, cade oil, calamus oil, camphor oil, cananga oil, cardamom oil, cascarilla oil, cassia oil, cassie absolute, castoreum absolute, cedar leaf oil, cedar wood oil, cistus oil, citronella oil, lemon oil, copaiba balsam, copaiba balsam oil, coriander oil, costus root oil, cumin oil, cypress oil, davana oil, dill weed oil, dill seed oil, eau de brouts absolute, oakmoss absolute, elemi oil, estragon oil, eucalyptus citriodora oil, eucalyptus oil (cineole type), fennel oil, fir needle oil, galbanum oil, galbanum resin, geranium oil, grapefruit oil, guaiacwood oil, gurjun balsam, gurjun balsam oil, helichrysum absolute, helichrysum oil, ginger oil, iris root absolute, iris root oil, jasmine absolute, calamus oil, blue chamomile oil, Roman chamomile oil, carrot seed oil, cascarilla oil, pine needle oil, spearmint oil, caraway oil, labdanum oil, labdanum absolute, labdanum resin, lavandin absolute, lavandin oil, lavender absolute, lavender oil, lemon-grass oil, lovage oil, lime oil distilled, lime oil expressed, linaloe oil, Litsea cubeba oil, laurel leaf oil, mace oil, marjoram oil, mandarin oil, massoi (bark) oil, mimosa absolute, ambrette seed oil, musk tincture, clary sage oil, nutmeg oil, myrrh absolute, myrrh oil, myrtle oil, clove leaf oil, clove bud oil, neroli oil, olibanum absolute, olibanum oil, opopanax oil, orange flower absolute, orange oil, origanum oil, palmarosa oil, patchouli oil, perilla oil, Peru balsam oil, parsley leaf oil, parsley seed oil, petitgrain oil, peppermint oil, pepper oil, pimento oil, pine oil, pennyroyal oil, rose absolute, rosewood oil, rose oil, rosemary oil, Dalmatian sage oil, Spanish sage oil, sandal-wood oil, celery seed oil, spike-lavender oil, star anise oil, storax oil, tagetes oil, fir needle oil, tea tree oil, turpentine oil, thyme oil, Tolu balsam, tonka bean absolute, tuberose absolute, vanilla extract, violet leaf absolute, verbena oil, vetiver oil, juniperberry oil, wine lees oil, wormwood oil, wintergreen oil, ylang-ylang oil, hyssop oil, civet absolute, cinnamon leaf oil, cinnamon bark oil, and fractions thereof or ingredients isolated therefrom;
(ii) hydrocarbons such as, for example, 3-carene, a-pinene, β-pinene, a-terpinene, γ-terpinene, p-cymene, bisabolene, camphene, caryophyllene, cedrene, farnesene, limonene, longifolene, myrcene, ocimene, valencene, (E,Z)-1,3,5-undecatriene, styrene, and diphenylmethane;
(iii) aliphatic alcohols such as, for example, hexanol, octanol, 3-octanol, 2,6-dimethyl-heptanol, 2-methyl-2-heptanol, 2-methyl-2-octanol, (E)-2-hexenol, (E)- and (Z)-3-hexenol, 1-octen-3-ol, a mixture of 3,4,5, 6,6-pentamethyl-3/4-hepten-2-ol and 3,5,6,6-tetramethyl-4-methyleneheptan-2-ol, (E,Z)-2,6-nonadienol, 3,7-dimethyl-7-methoxyoctan-2-ol, 9-decenol, 10-undecenol, 4-methyl-3-decen-5-ol, aliphatic aldehydes and their acetals such as for example hexanal, heptanal, octanal, nonanal, decanal, undecanal, dodecanal, tridecanal, 2-methyloctanal, 2-methylnonanal, (E)-2-hexenal, (Z)-4-heptenal, 2,6-dimethyl-5-heptenal, 10-undecenal, (E)-4-decenal, 2-dodecenal, 2,6,10-trimethyl-5,9-undecadienal, heptanal-diethylacetal, 1,1-dimethoxy-2,2,5-trimethyl-4-hexene, and citronellyl oxyacetaldehyde;
(iv) aliphatic ketones and oximes thereof, such as, for example, 2-heptanone, 2-octanone, 3-octanone, 2-nonanone, 5-methyl-3-heptanone, 5-methyl-3-heptanone oxime, 2,4,4,7-tetra-methyl-6-octen-3-one, aliphatic sulfur-containing compounds, such as for example 3-methylthio-hexanol, 3-methylthiohexyl acetate, 3-mercaptohexanol, 3-mercaptohexyl acetate, 3-mercapto-hexyl butyrate, 3-acetylthiohexyl acetate, 1-menthene-8-thiol, and aliphatic nitriles (e.g., 2-nonenenitrile, 2-tridecenenitrile, 2,12-tridecenenitrile, 3,7-dimethyl-2,6-octadienenitrile, and 3,7-dimethyl-6-octenenitrile);
(v) aliphatic carboxylic acids and esters thereof, such as, for example, (E)- and (Z)-3-hexenylformate, ethyl acetoacetate, isoamyl acetate, hexyl acetate, 3,5,5-trimethylhexyl acetate, 3-methyl-2-butenyl acetate, (E)-2-hexenyl acetate, (E)- and (Z)-3-hexenyl acetate, octyl acetate, 3-octyl acetate, l-octen-3-yl acetate, ethyl butyrate, butyl butyrate, isoamyl butyrate, hexylbutyrate, (E)- and (Z)-3-hexenyl isobutyrate, hexyl crotonate, ethylisovalerate, ethyl-2-methyl pentanoate, ethyl hexanoate, allyl hexanoate, ethyl heptanoate, allyl heptanoate, ethyl octanoate, ethyl-(E,Z)-2,4-decadienoate, methyl-2-octinate, methyl-2-noninate, allyl-2-isoamyl oxyacetate, and methyl-3,7-dimethyl-2,6-octadienoate; (vi) acyclic terpene alcohols, such as, for example, citronellol, geraniol, nerol, linalool, lavandulol, nerolidol, farnesol, tetrahydrolinalool, tetrahydrogeraniol, 2,6-dimethyl-7-octen-2-ol, 2,6-dimethyloctan-2-ol, 2-methyl-6-methylene-7-octen-2-ol, 2,6-dimethyl-5,7-octadien-2-ol, 2,6-dimethyl-3,5-octadien-2-ol, 3,7-dimethyl-4,6-octadien-3-ol, 3,7-dimethyl-1,5,7-octatrien-3-ol, 2,6-dimethyl-2,5,7-octatrien-1-ol, as well as formates, acetates, propionates, isobutyrates, butyrates, isovalerates, pentanoates, hexanoates, crotonates, tiglinates and 3-methyl-2-butenoates thereof;
(vii) acyclic terpene aldehydes and ketones, such as, for example, geranial, neral, citronellal, 7-hydroxy-3,7-dimethyloctanal, 7-methoxy-3,7-dimethyloctanal, 2,6,10-trimethyl-9-undecenal, α-sinensal, β-sinensal, geranylacetone, as well as the dimethyl- and diethylacetals of geranial, neral and 7-hydroxy-3,7-dimethyloctanal;
(viii) cyclic terpene alcohols, such as, for example, menthol, isopulegol, alpha-terpineol, terpinen-4-ol, menthan-8-ol, menthan-l-ol, menthan-7-ol, borneol, isoborneol, linalool oxide, nopol, cedrol, ambrinol, vetiverol, guaiol, and the formates, acetates, propionates, isobutyrates, butyrates, isovalerates, pentanoates, hexanoates, crotonates, tiglinates and 3-methyl-2-butenoates of alpha-terpineol, terpinen-4-ol, methan-8-ol, methan-1-ol, methan-7-ol, borneol, isoborneol, linalool oxide, nopol, cedrol, ambrinol, vetiverol, and guaiol;
(ix) cyclic terpene aldehydes and ketones, such as, for example, menthone, isomenthone, 8-mercaptomenthan-3-one, carvone, camphor, fenchone, α-ionone, β-ionone, α-n-methylionone, β-n-methylionone, α-isomethylionone, β-isomethylionone, α-irone, α-damascone, β-damascone, β-damascenone, δ-damascone, γ-damascone, 1-(2,4,4-trimethyl-2-cyclohexen-1-yl)-2-buten-1-one, 1,3,4,6,7,8a-hexahydro-1,1,5,5-tetramethyl-2H-2,4a-methanonaphthalen-8(5H-)-one, nootkatone, dihydronootkatone; acetylated cedarwood oil (cedryl methyl ketone);
(x) cyclic alcohols, such as, for example, 4-tert-butylcyclohexanol, 3,3,5-trimethylcyclo-hexanol, 3-isocamphylcyclohexanol, 2,6,9-trimethyl-Z2,Z5,E9-cyclododecatrien-1-ol, 2-iso-butyl-4-methyltetrahydro-2H-pyran-4-ol;
(xi) cycloaliphatic alcohols, such as, for example, alpha, 3,3-trimethylcyclo-hexylmethanol, 2-methyl-4-(2,2,3-trimethyl-3-cyclopent-1-yl)butanol, 2-methyl-4-(2,2,3-trimethyl-3-cyclopent-1-yl)-2-buten-1-ol, 2-ethyl-4-(2,2,3-trimethyl-3-cyclopent-1-yl)-2-buten-1-ol, 3-methyl-5-(2,2,3-trimethyl-3-cyclopent-1-yl)-pentan-2-ol, 3-methyl-5-(2,2,3-trimethyl-3-cyclopent-1-yl)-4-penten-2-ol, 3,3-dimethyl-5-(2,2,3-trimethyl-3-cyclopent-1-yl)-4-penten-2-ol, 1-(2,2,6-trimethylcyclohexyl)pentan-3-ol, 1-(2,2,6-trimethylcyclohexyl)hexan-3-ol;
(xii) cyclic and cycloaliphatic ethers, such as, for example, cineole, cedryl methyl ether, cyclododecyl methyl ether;
(xiii) (ethoxymethoxy)cyclododecane; alpha-cedrene epoxide, 3a,6,6,9a-tetramethyl-dodecahydronaphtho[2,1-b]furan, 3a-ethyl-6,6,9a-trimethyldodecahydronaphtho[2,1-b]furan, 1,5,9-trimethyl-13-oxabicyclo[10.1.0]-trideca-4,8-diene, rose oxide, 2-(2,4-dimethyl-3-cyclohexen-1-yl)-5-methyl-5-(1-methylpropyl)-1,3-dioxan;
(xiv) cyclic ketones, such as, for example, 4-tert-butylcyclohexanone, 2,2,5-trimethyl-5-pentylcyclopentanone, 2-heptylcyclopentanone, 2-pentylcyclopentanone, 2-hydroxy-3-methyl-2-cyclopenten-1-one, 3-methyl-cis-2-penten-1-yl-2-cyclopenten-1-one, 3-methyl-2-pentyl-2-cyclopenten-1-one, 3-methyl-4-cyclopentadecenone, 3-methyl-5-cyclopentadecenone, 3-methylcyclopentadecanone, 4-(1-ethoxyvinyl)-3,3,5,5-tetramethylcyclohexanone, 4-tert-pentylcyclohexanone, 5-cyclohexadecen-1-one, 6,7-dihydro-1,1,2,3,3-pentamethyl-4(5H)-indanone, 5-cyclohexadecen-1-one, 8-cyclohexadecen-1-one, 9-cycloheptadecen-1-one, cyclopentadecanone, cycloaliphatic aldehydes, such as, for example, 2,4-dimethyl-3-cyclohexene carbaldehyde, 2-methyl-4-(2,2,6-trimethyl-cyclohexen-1-yl)-2-butenal, 4-(4-hydroxy-4-methylpentyl)-3-cyclohexene carbaldehyde, 4-(4-methyl-3-penten-1-yl)-3-cyclohexene carbaldehyde;
(xv) cycloaliphatic ketones, such as, for example, 1-(3,3-dimethylcyclohexyl)-4-penten-1-one, 1-(5,5-dimethyl-1-cyclohexen-1-yl)-4-penten-1-one, 2,3,8,8-tetramethyl-1,2,3,4,5,6,7,8-octahydro-2-naphtalenyl methyl-ketone, methyl-2,6,10-trimethyl-2,5,9-cyclododecatrienyl ketone, tert-butyl-(2,4-dimethyl-3-cyclohexen-1-yl)ketone;
(xvi) esters of cyclic alcohols, such as, for example, 2-tert-butylcyclohexyl acetate, 4-tert-butylcyclohexyl acetate, 2-tert-pentylcyclohexyl acetate, 4-tert-pentylcyclohexyl acetate, decahydro-2-naphthyl acetate, 3-pentyltetrahydro-2H-pyran-4-yl acetate, decahydro-2,5,5,8a-tetramethyl-2-naphthyl acetate, 4,7-methano-3a,4,5,6,7,7a-hexahydro-5 or 6-indenyl acetate, 4,7-methano-3a,4,5,6,7,7a-hexahydro-5 or 6-indenyl propionate, 4,7-methano-3a,4,5,6,7,7a-hexahydro-5 or 6-indenyl-isobutyrate, 4,7-methanooctahydro-5 or 6-indenyl acetate;
(xvii) esters of cycloaliphatic carboxylic acids, such as, for example, allyl 3-cyclohexyl-propionate, allyl cyclohexyl oxyacetate, methyl dihydrojasmonate, methyl jasmonate, methyl 2-hexyl-3-oxocyclopentanecarboxylate, ethyl 2-ethyl-6,6-dimethyl-2-cyclohexenecarboxylate, ethyl 2,3,6,6-tetramethyl-2-cyclohexenecarboxylate, ethyl 2-methyl-1,3-dioxolane-2-acetate;
(xviii) aromatic and aliphatic alcohols, such as, for example, benzyl alcohol, 1-phenylethyl alcohol, 2-phenylethyl alcohol, 3-phenylpropanol, 2-phenylpropanol, 2-phenoxyethanol, 2,2-dimethyl-3-phenylpropanol, 2,2-dimethyl-3-(3-methylphenyl)propanol, 1,1-dimethyl-2-phenylethyl alcohol, 1,1-dimethyl-3-phenylpropanol, 1-ethyl-1-methyl-3-phenylpropanol, 2-methyl-5-phenylpentanol, 3-methyl-5-phenylpentanol, 3-phenyl-2-propen-1-ol, 4-methoxybenzyl alcohol, 1-(4-isopropylphenyl)ethanol;
(xix) esters of aliphatic alcohols and aliphatic carboxylic acids, such as, for example, benzyl acetate, benzyl propionate, benzyl isobutyrate, benzyl isovalerate, 2-phenylethyl acetate, 2-phenylethyl propionate, 2-phenylethyl isobutyrate, 2-phenylethyl isovalerate, 1-phenylethyl acetate, α-trichloromethylbenzyl acetate, α,α-dimethylphenylethyl acetate, α,α-dimethylphenylethyl butyrate, cinnamyl acetate, 2-phenoxyethyl isobutyrate, 4-methoxybenzyl acetate, araliphatic ethers, such as for example 2-phenylethyl methyl ether, 2-phenylethyl isoamyl ether, 2-phenylethyl-1-ethoxyethyl ether, phenylacetaldehyde dimethyl acetal, phenylacetaldehyde diethyl acetal, hydratropaaldehyde dimethyl acetal, phenylacetaldehyde glycerol acetal, 2,4,6-trimethyl-4-phenyl-1,3-dioxane, 4,4a,5,9b-tetrahydroindeno[1,2-d]-m-dioxin, 4,4a,5,9b-tetrahydro-2,4-dimethylindeno[1,2-d]-m-dioxin;
(xx) aromatic and aliphatic aldehydes, such as, for example, benzaldehyde; phenylacetaldehyde, 3-phenylpropanal, hydratropaldehyde, 4-methylbenzaldehyde, 4-methylphenyl-acetaldehyde, 3-(4-ethylphenyl)-2,2-dimethylpropanal, 2-methyl-3-(4-isopropylphenyl)propanal, 2-methyl-3-(4-tert-butylphenyl)propanal, 3-(4-tert-butylphenyl)propanal, cinnamaldehyde, α-butylcinnamaldehyde, α-amylcinnamaldehyde, α-hexylcinnamaldehyde, 3-methyl-5-phenylpentanal, 4-methoxybenzaldehyde, 4-hydroxy-3-methoxybenzaldehyde, 4-hydroxy-3-ethoxybenzaldehyde, 3,4-methylene-dioxybenzaldehyde, 3,4-dimethoxybenzaldehyde, 2-methyl-3-(4-methoxyphenyl)propanal, 2-methyl-3-(4-methylendioxyphenyl)propanal;
(xxi) aromatic and aliphatic ketones, such as, for example, acetophenone, 4-methylaceto-phenone, 4-methoxyacetophenone, 4-tert-butyl-2,6-dimethylacetophenone, 4-phenyl-2-butanone, 4-(4-hydroxyphenyl)-2-butanone, 1-(2-naphthalenyl)ethanone, benzophenone, 1,1,2,3,3,6-hexamethyl-5-indanyl methyl ketone, 6-tert-butyl-1,1-dimethyl-4-indanyl methyl ketone, 1-[2,3-dihydro-1,1,2,6-tetramethyl-3-(1-methyl-ethyl)-1H-5-indenyl]ethanone, 5′,6′,7′,8′-tetrahydro-3′,5′,5′,6′,8′,8′-hexamethyl-2-acetonaphthone;
(xxii) aromatic and araliphatic carboxylic acids and esters thereof, such as, for example, benzoic acid, phenylacetic acid, methyl benzoate, ethyl benzoate, hexyl benzoate, benzyl benzoate, methyl phenylacetate, ethyl phenylacetate, geranyl phenylacetate, phenylethyl phenylacetate, methyl cinnamate, ethyl cinnamate, benzyl cinnamate, phenylethyl cinnamate, cinnamyl cinnamate, allyl phenoxyacetate, methyl salicylate, isoamyl salicylate, hexyl salicylate, cyclohexyl salicylate, cis-3-hexenyl salicylate, benzyl salicylate, phenylethyl salicylate, methyl 2,4-dihydroxy-3,6-dimethylbenzoate, ethyl 3-phenylglycidate, ethyl 3-methyl-3-phenylglycidate;
(xxiii) nitrogen-containing aromatic compounds, such as, for example, 2,4,6-trinitro-1,3-dimethyl-5-tert-butylbenzene, 3,5-dinitro-2,6-dimethyl-4-tert-butylacetophenone, cinnamonitrile, 5-phenyl-3-methyl-2-pentenonitrile, 5-phenyl-3-methylpentanonitrile, methyl anthranilate, methyl-N-methylanthranilate, Schiffs bases of methyl anthranilate with 7-hydroxy-3,7-dimethyl-octanal, 2-methyl-3-(4-tert-butylphenyl)propanal or 2,4-dimethyl-3-cyclohexene carbaldehyde, 6-isopropylquinoline, 6-isobutylquinoline, 6-sec-butylquinoline, indole, skatole, 2-methoxy-3-isopropylpyrazine, 2-isobutyl-3-methoxypyrazine;
(xxiv) phenols, phenyl ethers and phenyl esters, such as, for example, estragole, anethole, eugenol, eugenyl methyl ether, isoeugenol, isoeugenol methyl ether, thymol, carvacrol, diphenyl ether, S-naphthyl methyl ether, S-naphthyl ethyl ether, S-naphthyl isobutyl ether, 1,4-dimethoxybenzene, eugenyl acetate, 2-methoxy-4-methylphenol, 2-ethoxy-5-(1-propenyl)phenol, p-cresyl phenylacetate;
(xxv) heterocyclic compounds, such as, for example, 2,5-dimethyl-4-hydroxy-2H-furan-3-one, 2-ethyl-4-hydroxy-5-methyl-2H-furan-3-one, 3-hydroxy-2-methyl-4H-pyran-4-one, 2-ethyl-3-hydroxy-4H-pyran-4-one;
(xxvi) lactones, such as, for example, 1,4-octanolide, 3-methyl-1,4-octanolide, 1,4-nonanolide, 1,4-decanolide, 8-decen-1,4-olide, 1,4-undecanolide, 1,4-dodecanolide, 1,5-decanolide, 1,5-dodecanolide, 1,15-pentadecanolide, cis- and trans-11-pentadecen-1,15-olide, cis- and trans-12-pentadecen-1,15-olide, 1,16-hexadecanolide, 9-hexadecen-1,16-olide, 10-oxa-1,16-hexadecanolide, 11-oxa-1,16-hexadecanolide, 12-oxa-1,16-hexadecanolide, ethylene-1,12-dodecanedioate, ethylene-1,13-tridecanedioate, coumarin, 2,3-dihydrocoumarin, and octahydrocoumarin; and
(xxvii) malodor counteracting agents including an α,β-unsaturated carbonyl compounds including but not limited to those disclosed in U.S. Pat. No. 6,610,648 and EP 2,524,704, amyl cinnamaldehyde, benzophenone, benzyl benzoate, benzyl isoeugenol, benzyl phenyl acetate, benzyl salicylate, butyl cinnamate, cinnamyl butyrate, cinnamyl isovalerate, cinnamyl propionate, decyl acetate, ethyl myristate, isobutyl cinnamate, isoamyl salicylate, phenethyl benzoate, phenethyl phenyl acetate, triethyl citrate, tripropylene glycol n-butyl ether, isomers of bicyclo[2.2.1]hept-5-ene-2-carboxylic acid, ethyl ester, nano silver, zinc undecenylate, β-naphthyl methyl ether, β-naphthyl ketone, benzyl acetone. They may include mixture of hexahydro-4,7-methanoinden-5-yl propionate and hexahydro-4,7-methanoinden-6-yl propionate; 4-(2,6,6-trimethyl-2-cyclohexen-1-yl)-3-methyl-3-buten-2-one; 3,7-dimethyl-2,6-nonadien-1-nitrile; dodecahydro-3a, 6,6,9a-tetramethylnaphtho (2,1-b) furan; ethylene glycol cyclic ester of n-dodecanedioic acid; 1-cyclohexadecen-6-one; 1-cycloheptadecen-10-one; and corn mint oil. They may also include 1-cyclohexylethan-1-yl butyrate; 1-cyclohexylethan-1-yl acetate; 1-cyclohexylethan-1-ol; 1-(4′-methylethyl)cyclohexylethan-1-yl propionate; and 2′-hydroxy-1′-ethyl(2-phenoxy)acetate each of which compound is marketed under the trademark VEILEX by International Flavors & Fragrances Inc. More suitable malodor counteracting agents are polymers containing an α-keto, benzaldehyde, or α,β-unsaturated carbonyl moiety, such as those described in US 2012/0294821, US 2013/0101544 and US 2013/0101545.

Additional fragrance ingredients are described in, e.g., Arctander (2000) Perfume Flavors and Chemicals. Vols. I and II, Montclair, NJ; The Merck Index, 8^{th} Edition (1968) Merck & Co., Inc., Rahway, NJ; Allured's Flavor and Fragrance Materials (2015) Allured Publishing Corp.

In accordance with the present invention, a fragrance composition of this invention is composed of between 3 to 30 wt. %, such as between 3 to 25 wt. %, between 5 to 30 wt. %, or between 5 to 25 wt. % or between 5 wt. % and 20 wt. % fragrance ingredient relative to the total weight of the fragrance composition; between 5 wt. % and 30 wt. % (preferably between 15 wt. % and 20 wt. %) solubilizer component relative to the total weight of the fragrance composition; between 3 wt. % and 18 wt. % (preferably between 5 wt. % and 10 wt. %) emollient component relative to the total weight of the fragrance composition; and between 40 wt. % and 95 wt. % (preferably between 40 wt. % and 60 wt. %) solvent component relative to the total weight of the fragrance composition.

In some aspects, the fragrance composition is composed of between 3 to 30 wt. %, such as between 3 to 25 wt. %, between 5 to 30 wt. %, or between 5 to 25 wt. % or between 5 wt. % and 20 wt. % fragrance ingredient relative to the total weight of the fragrance composition; between 5 wt. % and 30 wt. % solubilizer component relative to the total weight of the fragrance composition; between 2 wt. % and 15 wt. % isopentyldiol relative to the total weight of the fragrance composition; between 1 wt. % and 8 wt. % 3-methoxy-3-methyl-1-butanol relative to the total weight of the fragrance composition; between 25 wt. % and 60 wt. % water relative to the total weight of the fragrance composition; and between 15 wt. % and 35 wt. % pentylene glycol relative to the total weight of the fragrance composition.

In other aspects, the fragrance composition of this invention is formulated as a consumer product. Consumer products including the fragrance composition of this invention include, e.g., a fine fragrance, a solid perfume, eau de parfum, parfum, cologne, alcohol-free perfume, perfume powder, perfume wax, perfume emulsion, a powder foundation, or an eau de toilette product, deodorant, air freshener, room or laundry spray, preferably an eau de toilette, room or laundry spray. Preferably, the consumer product of the invention is not in the form of cream or deodorant stick.

The consumer product can contain any fragrance composition described herein at a level of 0.01 wt. % to 100 wt. % (e.g., 0.02 wt. % to 10 wt. %, 0.05 wt. % to 5 wt. %, 0.05 wt. % to 2 wt. %, 0.1 wt. % to 0.5 wt. %), wherein the lower limit may be 0.01 wt. %, 0.02 wt. %, 0.05 wt. %, 0.1 wt. %, 0.2 wt. %, or 0.5 wt. %, and the upper limit may be 100 wt. %, 90 wt. %, 50 wt. %, 20 wt. %, 10 wt. %, 5 wt. %, 3 wt. %, 2 wt. %, 1 wt. %, 0.8 wt. %, or 0.5 wt. %.

The fragrance composition, and/or consumer product can further include one or more preservatives, colorants, dyes, hydrophobic light-stability agents, lipophilic light-stability agents, antifoaming agents, thickeners, complexing agents, antioxidants, light protection filters, stabilizers such as radical absorbers and/or substances that have an additional benefit for the user, for example cosmetic active substances.

The invention further provides a method for preparing a fragrance composition by mixing at least one fragrance ingredient with the fragrance base, in particular a fragrance base including the at least one solubilizer, the at least two emollients, and the at least two solvents described herein, in any order (or simultaneously). In certain aspects, the invention provides a method for preparing a fragrance composition my mixing at least one fragrance with at least one solubilizer, said solubilizer being selected from the group of polysorbate 20, sodium olivate, Glycereth Cocoate and polyethylene glycol (16) macadamia glycerides; at least two emollients, said emollients comprising isopentyldiol and 3-methoxy-3-methyl-1-butanol; and at least two solvents, said solvents comprising water and pentylene glycol.

All parts, percentages and proportions refer to herein and in the claims are by weight unless otherwise indicated.

The values and dimensions disclosed herein are not to be understood as being strictly limited to the exact numerical values recited. Instead, unless otherwise specified, each such value is intended to mean both the recited value and a functionally equivalent range surrounding that value. For example, a value disclosed as “50%” is intended to mean “about 50%” and encompasses, e.g., 50±5%.

When an amount, concentration, or other value or parameter is given as either a range, preferred range or a list of upper preferable values and/or lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. For example, when a range of “1 to 10” is recited, the recited range should be construed as including ranges “1 to 8,” “3 to 10,” “2 to 7,” “1.5 to 6,” “3.4 to 7.8,” “1 to 2 and 7-10,” “2 to 4 and 6 to 9,” “1 to 3.6 and 7.2 to 8.9,” “1-5 and 10,” “2 and 8 to 10,” “1.5-4 and 8,” and the like.

The terms “g,” “mg,” and “μg” refer to “gram,” “milligram,” and “microgram,” respectively. The terms “L” and “mL” refer to “liter” and “milliliter,” respectively.

Without further elaboration, it is believed that one skilled in the art can, based on the description herein, utilize the present invention to its fullest extent. All publications cited herein are incorporated by reference in their entirety.

### Example 1: Base Formula with Fragrance Added

The alcohol-free base of the invention was formulated with dipropylene glycol (DPG) and 11% fragrance (Table 1).

TABLE 1

| Ingredient | Function | Wt % |
|---|---|---|
| Purified Water | Diluent/solvent | 38 |
| DPG | Diluent/solvent | 3 |
| Isoprene Glycol | Emollient | 8 |
| MMB | Emollient | 5 |
| Polysorbate 20 | Solubilizer | 15 |
| Pentylene Glycol | Solubilizer/Preservative | 20 |
| Fragrance | Fragrance | 11 |

MMB, 3-methoxy-3-methyl-1-butanol.

### Example 2: Sensory Performance of Alcohol-Free Base Formula

Fragrances (11 wt %) in the alcohol-free base of Table 1 (Samples 1 and 2) were compared to a similar fragrance (16 wt %) in a hydroalcoholic base (Comparative: 80 wt % specially denatured alcohol 40B and 4 wt % water). Fragrances for Samples 1 and 2 were similar to one another, with Sample 1 being an original fragrance and Sample 2 having fewer high log P musks.

The fragrance formulations were applied to a substrate and allowed to dry for 4 hours. A trained panel of subjects (N=12) conducted a blind and randomized sensory analysis of the fresh and dried samples. The panel assessed intensity, i.e., the impact and substantivity of the fragrances, on an 11 point scale at 0 hour and 4 hours after application. In addition, the panel assessed the ability of the fragrance in each sample to maintain its character. The analysis was based upon a 7-point scale (very different→identical) in a paired comparison (percentage of lasting intensity after 4 hours: calculated slope between T₀h and T₄h).

This analysis indicated that while there was no statistically significant difference in impact or substantivity when comparing the two fragrances in the alcohol-free base as compared to a similar fragrance in a hydroalcoholic base, the alcohol-free base exhibited statistically better longevity (Table 2).

TABLE 2

Sample

| Sensory Measurement | Sample 1 | Sample 2 | Comparative |
|---|---|---|---|
| Impact (T₀h) | 6.77^{a} | 6.82^{a} | 6.93^{a} |
| Substantivity (T₄h) | 6.26^{a} | 6.17^{a} | 5.94^{a} |
| Lastingness | 93.01^{a} | 91.18^{ab} | 86.19^{b} |

Samples not connected by same letter are significantly different. Significance level=10%.

### Example 3: Environmentally Friendly Alcohol-Free Base Formula

Further to the examples above, a number of “green” or environmentally friendly formulations enabling alcohol-free base were tested (Table 3).

TABLE 3

| Ingredient | Function | 3% | 11% | 25% | comment |
|---|---|---|---|---|---|
| Sodium | Solubilizer | 6.75 | 11.25 | 18 | replaces |
| olivate | | | | | Polysorbate |
| | | | | | 20 |
| Glycereth | Solubilizer | 2.25 | 3.75 | 6 | replaces |
| Cocoate | | | | | Polysorbate |
| | | | | | 20 |
| Fragrance oil | Fragrance | 3 | 11 | 25 | |
| Butylene | Diluent/ | 9 | 9 | 7.5 | replaces |
| glycol | solvent | | | | DPG |
| Isoprene | Emollient | 2 | 2 | 2 | |
| Glycol | | | | | |
| MMB | Emollient | 5 | 5 | 1.75 | |
| Pentylene | Solubilizer/ | 20 | 20 | 16 | |
| Glycol | Preservative | | | | |
| Water | Diluent/ | 52 | 38 | 23.75 | |
| | solvent | | | | |
| Total | | 100 | 100 | 100 | |

MMB, 3-methoxy-3-methyl-1-butanol
DPG, dipropylene glycol

Specifically, the solubilizers sodium olivate and Glycereth Cocoate can replace Polysorbate 20 and the diluent/solvent butylene glycol can replace DPG.

The environmentally friendly formulations of Table 3 passed stability tests with retaining clarity and olfactive intensity and character. The test conditions include freeze-thaw cycles, and accelerated testing at 5° C., 25° C., 45° C. (data not shown).

It is particularly interesting that high levels of fragrance (>15%) can be used with the formulations with retained properties.

The range of recited numerical values disclosed in the specification includes values, e.g., +/−5-10% of the recited value, that a person of ordinary skill in the art would consider equivalent to the recited value, e.g., having the same function or result.

The claims are not limited by the preferred embodiments and examples but will cover many modifications and equivalents consistent with the written description as a whole.

1. A transparent alcohol-free base comprising:
(i) at least one solubilizer, said solubilizer being selected from the group of polysorbate 20, sodium olivate and Glycereth Cocoate;
(ii) at least two emollients, said emollients comprising isopentyldiol and 3-methoxy-3-methyl-1-butanol; and
(iii) at least two solvents, said solvents being selected from the group of water, butylene glycol and pentylene glycol.

2. A fragrance composition comprising the transparent alcohol-free base of claim 1 and at least one fragrance ingredient.

3. The fragrance composition of claim 2, wherein the fragrance composition comprises:
(i) 3 to 30 wt. % of at least one fragrance ingredient;
(ii) 5 to 30 wt. % at least one solubilizer, said solubilizer being selected from the group of polysorbate 20, sodium olivate and Glycereth Cocoate;
(iii) 3 to 23 wt. % at least two emollients, said emollients comprising isopentyldiol and 3-methoxy-3-methyl-1-butanol; and
(iv) 40 to 95 wt. % at least two solvents, said solvents being selected from the group of water, butylene glycol and pentylene glycol.

4. The fragrance composition of claim 2, wherein the fragrance composition comprises:
(i) 3 to 30 wt. % of at least one fragrance ingredient;
(ii) 5 to 30 wt. % polysorbate 20, sodium olivate or Glycereth Cocoate;
(iii) 2 to 15 wt. % isopentyldiol;
(iv) 1 to 8 wt. % 3-methoxy-3-methyl-1-butanol;
(v) 15 to 35 wt. % pentylene glycol; and
(vi) 25 to 60 wt. % water.

5. The fragrance composition of claim 2, comprising between 3 to 30 wt. %, between 3 to 25 wt. %, between 5 to 30 wt. %, or between 5 to 25 wt. % fragrance ingredient.

6. A consumer product comprising the fragrance composition of claim 2.

7. The consumer product of claim 6, said consumer product being selected from the group consisting of a fine fragrance, solid perfume, eau de parfum, parfum, cologne, alcohol-free perfume, perfume powder, perfume wax, perfume emulsion, powder foundation, or eau de toilette product, deodorant, air freshener, room spray or laundry spray.

8. A method for preparing a fragrance composition comprising mixing at least one fragrance ingredient with the transparent alcohol-free base of claim 1 thereby preparing a fragrance composition.
